(19) United States
(12) Patent Application Publication
(10) Pub. No.: US 2009/0137175 A1
(43) Pub. Date: May 28, 2009

(54) Sizing Composition For Glass Fibers, Sized Glass Fibers, And Reinforced Products Comprising The Same

(76) Inventors: Jacobus Hendricus Antonius Van Der Woude, Leek (NL); Jacob Cornelius Dijt, Eelde (NL); John Theo Penning, Tynaarlo (NL); Ronald Boelman, Stadskanaal (NL); Johannes Leonardus Tabak, Groningen (NL)

Correspondence Address: KILPATRICK STOCKTON LLP, 1001 WEST FOURTH STREET, WINSTON-SALEM, NC, 27101, US

(21) Appl. No.: 12/267,963

(22) Filed: Nov. 10, 2008

Related U.S. Application Data
(60) Provisional application No. 61/002,370, filed on Nov. 8, 2007.

(51) Int. Cl. B32B 17/02; C07F 9/28; C07C 309/00; B32B 17/10; C07D 233/04; C07F 7/02

(52) U.S. Cl. 442/187; 562/8; 562/30; 548/347.1; 556/413; 428/392; 428/300.1; 442/331; 106/287.25; 524/115; 524/155

(57) ABSTRACT

The present invention provides aqueous sizing compositions for application to glass fibers as well as polymeric resins reinforced with glass fibers at least partially coated with the aqueous sizing compositions. In some embodiments, sizing compositions of the present invention demonstrate advantageous properties resulting from the presence of an acid-amine component therein.

### RELATED APPLICATION DATA

The present application hereby claims priority under 35 U.S.C. § 119(e) to U.S. Provisional Patent Application Ser. No. 61/002,370 filed Nov. 8, 2007, which is hereby incorporated by reference in its entirety.

### FIELD OF THE INVENTION

The present invention relates to sizing compositions for glass fibers, sized glass fibers, and articles reinforced with sized glass fibers.

### BACKGROUND OF THE INVENTION

A sizing composition operable to impart desired properties is typically applied to glass fibers subsequent to glass fiber formation. As used herein, the terms “sizing composition,” “sizing,” “binder composition,” “binder,” or “size” refer to a coating composition applied to the filaments after forming. In some embodiments, a sizing composition is applied to the filaments immediately after forming. Sizing compositions may provide protection through subsequent processing steps, such as those where the fibers pass by contact points as in the winding of the fibers and strands onto a forming package, drying the aqueous-based or solvent-based sizing composition to remove the water or solvent, twisting from one package to a bobbin, beaming to place the yarn onto very large packages ordinarily used as the warp in a fabric, chopping in a wet or dry condition, roving into larger bundles or groups of strands, unwinding for use as a reinforcement, and other downstream processes.

In addition, sizing compositions can play a dual role when placed on fibers that reinforce polymeric matrices in the production of composites and other products. In some applications, the sizing composition can provide protection and also can provide compatibility between the fiber and the matrix polymer or resin. For example, in some applications, glass fibers in the forms of woven and nonwoven fabrics, mats, rovings, and chopped strands have been compounded with resins, such as thermosetting and thermoplastic resins.

### SUMMARY

Embodiments of the present invention relate to aqueous sizing compositions for glass fibers. Moreover, embodiments of the present invention additionally relate to fiber glass strands comprising at least one glass fiber at least partially coated with an aqueous sizing composition of the present invention. Fiber glass strands at least partially coated with an aqueous sizing composition of the present invention can be used in encapsulation, impregnation, and reinforcement applications for a variety of resins, including thermoplastic and/or thermoset resins.

Embodiments of the present invention provide an aqueous sizing composition comprising an acid-amine component, the acid-amine component comprising molecules of at least one amine associated with molecules of at least one phosphorus-containing acid and/or sulfur containing acid.

In some embodiments, an amine of an acid-amine component comprises one or more amine chemical species generally used in sizing compositions for glass fibers including, but not limited to, aminosilane coupling agents and amine lubricants. In some embodiments, an amine of an acid-amine component comprises imidazolines, alkylimidazolines, ethoxylate amine oxides, polyamino fatty acid derivatives, or mixtures thereof.

Moreover, a phosphorus-containing acid of an acid-amine component, in some embodiments, comprises phosphorous acid, hypophosphorus acid, phosphonic acid, organophosphorus acids, phosphoric acid or mixtures thereof. Additionally, a sulfur-containing acid, in some embodiments, comprises sulfonic acid, organosulfonic acids, hydrogen sulfite, sulfurous acid, sulfuric acid or mixtures thereof. In some embodiments, a phosphorus-containing acid or a sulfur-containing acid can comprise the acid forms of the stabilizers set forth in U.S. Pat. No. 6,207,737 to Schell et al., which is hereby incorporated by reference.

Molecules of at least one amine, according to some embodiments of the present invention, are associated with molecules of at least one phosphorus-containing acid and/or sulfur-containing acid through electrostatic interactions, covalent bonds, dipole interactions, hydrogen bonds or van der Waals interactions or combinations thereof. In some embodiments, molecules of at least one phosphorus-containing acid and/or sulfur containing acid at least partially neutralize molecules of the at least one amine. As a result, phosphorus-containing acids and/or sulfur-containing acids, in some embodiments, can assist in controlling the pH of aqueous sizing compositions of the present invention.

In some embodiments of an aqueous sizing composition of the present invention, an acid-amine component comprises molecules of at least one amine associated with molecules of at least one phosphorus-containing acid and/or sulfur-containing acid and at least one additional acid. The at least one additional acid, in some embodiments, is selected to neutralize amine functionalities of the amine molecules which are not associated with the phosphorus-containing acid or sulfur-containing acid. In neutralizing amine functionalities not associated with the phosphorus-containing acid or sulfur-containing acid, the at least one additional acid can also assist in controlling the pH of the aqueous sizing composition.

In some embodiments, the at least one additional acid is selected according to the ability to neutralize amine functionalities and assist in providing the desired pH for the aqueous sizing composition. In one embodiment, the at least one additional acid comprises carboxylic acid. Carboxylic acids, according to some embodiments, can comprise alkanoic acids including, but not limited to, formic acid, acetic acid, propionic acid, butyric acid, valeric acid, caproic acid, enanthic acid, caprylic acid, pelargonic acid, capric acid, lauric acid, stearic acid, or mixtures thereof. Carboxylic acids, in other embodiments, can comprise unsaturated carboxylic acids, such as aromatic carboxylic acids, acrylic acids, or derivatives thereof. In some embodiments, carboxylic acids can comprise fatty acids or ketoacids such as a pyruvic acid and acetoacetic acid. In some embodiments, the at least one additional acid comprises carbonic acid, ascorbic acid, or mixtures thereof.

In another embodiment, the present invention provides a sizing composition comprising an acid-aminosilane component, the acid-aminosilane component comprising molecules of at least one aminosilane associated with molecules of at least one phosphorus-containing acid and/or sulfur containing acid. An aminosilane can comprise any aminosilane for use in sizing compositions known to one of skill in the art. In some embodiments, for example, an aminosilane can comprise aminopropyltrialkoxysilanes such as γ-aminopropyltrimethoxysilane and γ-aminopropyltriethoxysilane, β-aminoethyltriethoxysilane, N-β-aminoethylaminopropyltrimethoxysilane, 3-aminopropyldimethoxysilane, or mixtures thereof. Moreover, phosphorus-containing and sulfur-containing acids suitable for association with the at least one aminosilane are consistent with those described herein.

In some embodiments, an acid-aminosilane component comprises molecules of at least one aminosilane associated with molecules of at least one phosphorus-containing acid and/or sulfur containing acid and at least one additional acid. In some embodiments, at least one additional acid comprises carboxylic acids as described herein.

In some embodiments, an acid-amine component, such as an acid-aminosilane, can impart desirable film-forming properties to the sizing composition such that the amount of a separate film forming component can be eliminated or substantially reduced in the sizing composition. An acid-amine component can contribute to film build by providing higher molecular weight chemical species comprising molecules of at least one amine associated with molecules of at least one phosphorus-containing acid and/or sulfur-containing acid.

In some embodiments, nevertheless, a sizing composition of the present invention can further comprise at least one film former which supplements any film forming characteristics provided by the acid-amine component. In one embodiment, for example, a sizing composition comprises at least one film former and an acid-amine component, the acid-amine component comprising molecules of at least one amine associated with molecules of at least one phosphorus-containing acid and/or sulfur containing acid.

Alternatively, as acid-amine components can impart desirable film forming characteristics to sizing compositions of the present invention, in some embodiments, a sizing composition of the present invention does not comprise chemical species that primarily function as a film former. An acid-amine component can contribute to film build by providing higher molecular weight chemical species comprising molecules of at least one amine associated with molecules of at least one phosphorus-containing acid and/or sulfur-containing acid.

In some embodiments, sizing compositions of the present invention can further comprise one or more coupling agents, lubricants, biocides and/or anti-foaming agents. In some embodiments where the coupling agents and/or lubricants comprise amine functionalities, the coupling agents and/or lubricants are in addition to any aminosilane of aminofunctional lubricant participating in the formation of the acid-amine component.

Some embodiments of the present invention provide a fiber glass strand comprising at least one glass fiber at least partially coated with a sizing composition of the present invention. In some embodiments, a sizing composition of the present invention is a primary sizing. In other embodiments, a sizing composition of the present invention is a secondary sizing. The term “primary sizing” refers to a sizing composition applied to fibers immediately after formation of the fibers. The term “secondary sizing” refers to a composition applied to fibers after application of the primary sizing.

In some embodiments, for example, a sizing composition of the present invention comprising at least one film former and an acid-amine component is applied to at least one glass fiber as a primary sizing. A sizing composition comprising an acid-amine component without a separate film former is subsequently applied to the at least one glass fiber as a secondary sizing. Alternatively, in other embodiments, for example, a sizing composition of the present invention comprising an acid-amine component without a separate film former is applied to at least one glass fiber as a primary sizing, and a sizing composition of the present invention comprising an acid-amine component and at least one film former is subsequently applied to the at least one glass fiber as a secondary sizing.

Embodiments of the present invention contemplate use of any sizing composition described herein as a primary sizing or a secondary sizing. In some embodiments wherein a primary sizing comprises an acid-amine component, a secondary sizing subsequently applied does not comprise an acid-amine component. Moreover, in some embodiments wherein a secondary sizing comprises an acid-amine component, the primary sizing applied to a glass fiber does not comprise an acid-amine component.

Some embodiments of fiber glass strands comprising at least one fiber at least partially coated with a sizing composition of the present invention can comprise continuous strands or chopped strands. Moreover, continuous fiber glass strands, according to some embodiments, can be wound into a single package such as a forming package or a direct draw package.

In some embodiments, a plurality of fiber glass strands comprising at least one fiber at least partially coated with a sizing composition of the present invention are assembled into rovings.

In another aspect, the present invention provides fiber glass reinforced thermoplastic or thermoset composites. In some embodiments, a glass fiber reinforced thermoplastic or thermoset composite comprises a thermoplastic resin or thermoset resin and at least one glass fiber at least partially coated with a sizing composition of the present invention. As provided herein, glass fibers at least partially coated with a sizing composition of the present invention can have any desired length. In some embodiments, glass fibers at least partially coated with a sizing composition of the present invention have dimensions suitable for use in long fiber reinforcement applications including G-LFT (Granular-Long Fiber Thermoplastic) and D-LFT (Direct-Long Fiber Themoplastic).

Any desired thermoplastic or thermoset resin can be used with glass fibers at least partially coated with a sizing composition of the present invention. In some embodiments, thermoplastic resins reinforced with glass fibers of the present invention can comprise polyolefins such as polyethylene and polypropylene, polyamides, polyphenylene oxide, polystyrenics, or polyesters such as polybutylene terephthalate (PBT) and polyethylene terephthalate (PET). In some embodiments, thermoset resins reinforced with glass fibers of the present invention comprise polyester resins, polyimide resins, phenolic resins, and epoxy resins.

In a further embodiment, the present invention provides methods of making a glass fiber reinforced thermoplastic or thermoset composite. In one embodiment, a method of making a glass fiber reinforced thermoplastic or thermoset composite comprises providing a plurality of glass fibers, at least partially coating the plurality of glass fibers with a sizing composition of the present invention, and disposing the plurality of coated glass fibers in a thermoplastic or thermoset resin. As provided herein, glass fibers can be assembled into continuous fiber glass strands. The fiber glass strands can be chopped, remain continuous, or be assembled into rovings for reinforcement applications.

These and other embodiments are described in greater detail in the Detailed Description which follows.

### DETAILED DESCRIPTION

For the purposes of this specification, unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent.

Some embodiments of the present invention relate to new sizing compositions for glass fibers. The sizing compositions described herein generally relate to aqueous sizing compositions. Some embodiments of sizing compositions of the present invention are compatible with a variety of resins, including thermosetting resins, thermoplastic resins, and/or other polymeric resins. Some embodiments of the present invention also relate to fiber glass strands coated with sizing compositions. Additionally, some embodiments of the present invention relate to products that incorporate fiber glass strands, such as fiber reinforced polymeric materials.

The present invention will be discussed generally in the context of its use in the production, assembly, and application of glass fibers. However, one of ordinary skill in the art would understand that the present invention may be useful in the processing of other textile materials.

Some embodiments of fiber glass strands of the present invention can have various properties. For example, some embodiments of fiber glass strands can be at least partially coated with a sizing composition of the present invention resulting in the at least partially coated fiber glass strands being compatible with a number of resins while exhibiting acceptable levels of broken filaments, desirable abrasion resistance, and/or desirable strand integrity, as well as other properties.

In some embodiments, the present invention provides a sizing composition comprising an acid-amine component, the acid-amine component comprising molecules of at least one amine associated with molecules of at least one phosphorus-containing acid and/or sulfur-containing acid.

The amine of an acid-amine component, in some embodiments, comprises any amine having one or more amine groups operable to form an association with a phosphorus-containing acid or sulfur-containing acid. In some embodiments, an amine comprises a primary amine, secondary amine or tertiary amine or mixtures thereof.

In some embodiments, the amine of an acid-amine component comprises one or more amine chemical species generally used in sizing compositions for glass fibers including aminosilane coupling agents and amine lubricants. In some embodiments, the amine of an acid-amine component comprises aminosilanes, imidazolines, alkylimidazolines, ethoxylate amine oxides, polyethylenimine, polyamino fatty acid derivatives, or mixtures thereof. Suitable aminosilanes can comprise any aminosilane for use in sizing compositions known to one of skill in the art. In one embodiment, for example, an aminosilane can comprise aminopropyltrialkoxysilanes such as γ-aminopropyltrimethoxysilane and γ-aminopropyltriethoxysilane, β-aminoethyltriethoxysilane, N-β-aminoethylaminopropyltrimethoxysilane, 3-aminopropyldimethoxysilane, or mixtures thereof.

The acid of an acid-amine component, in some embodiments, can comprise a phosphorus-containing acid. The phosphorus atom of a phosphorus-containing acid, in some embodiments, can be in any one of oxidation states 3, 4, or 5. In other embodiments, the phosphorus atom of a phosphorus-containing acid is not in the highest oxidation state of 5. A phosphorus-containing acid, in some embodiments, comprises phosphorus acid, hypophosphorus acid, hypophosphoric acid, phosphonic acid, phosphoric acid or organophosphorus acids.

Organophosphorus acids suitable for use in some embodiments of the present invention can comprise those of Formula (I)

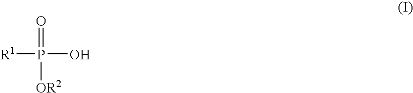

wherein R¹ is -alkyl, -alkenyl, -alkynyl, -cycloalkyl, -cycloalkenyl, -heterocycl, -aryl, or -heteroaryl and R² is -hydrogen, -alkyl, -alkenyl, -alkynyl, -cycloalkyl, -cycloalkenyl, -heterocycl, -aryl, or -heteroaryl.

In another embodiment, organophosphorus acids suitable for use in some embodiments of the present invention can comprise those of Formula (II)

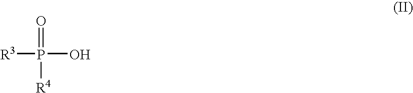

wherein R³ and R⁴ are independently selected from the group consisting of -hydrogen, -alkyl, -alkenyl, -alkynyl, -cycloalkyl, -cycloalkenyl, -heterocycl, -aryl, or -heteroaryl.

Organophosphorus acids, in some embodiments, can comprise phenylphosphinic acid, diphenylphosphinic acid, 4-methoxyphenyl phosphonic acid, 4-hydroxyphenyl phosphonic acid, 4-hydroxybutyl phosphonic acid, benzylhydrylphosphonic acid, benzylphosphonic acid, butylphosphonic acid, dodecylphosphonic acid, heptadecylphosphonic acid, methylbenzylphosphonic acid, naphthylmethylphosphonic acid, octadecylphosphonic acid, octylphosphonic acid, pentylphosphonic acid, phenylphosphonic acid, styrenephosphonic acid, or mixtures thereof.

In some embodiments, the acid of an acid-amine component can comprise a sulfur-containing acid. The sulfur atom of a sulfur-containing acid, in some embodiments, can be in any one of oxidation states 2, 4, or 6. In other embodiments, the sulfur atom of a sulfur-containing acid is not in the highest oxidation state of 6. A sulfur-containing acid, in one embodiment, comprises sulfonic acid, organosulfonic acids, hydrogen sulfite, sulfurous acid, sulfuric acid or mixtures thereof.

In some embodiments, an acid-amine component comprises molecules of at least one amine associated with molecules of at least one phosphorus-containing acid and/or sulfur-containing acid and at least one additional acid. In some embodiments, the at least one additional acid comprises a carboxylic acid as describer herein.

Molecules of at least one amine, according to some embodiments of the present invention, are associated with molecules of at least one phosphorus-containing acid and/or sulfur-containing acid through electrostatic interactions, covalent bonds, hydrogen bonds, dipole interactions or van der Waals interactions or combinations thereof. In one embodiment, for example, molecules of at least one amine are associated with molecules of at least one phosphorus-containing acid through electrostatic interactions as shown in Formula (III).

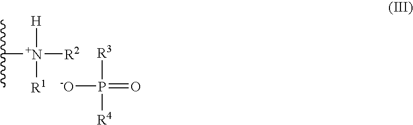

wherein R¹ through R⁴ are independently selected from the group consisting of -hydrogen, -alkyl, -alkenyl, -alkynyl, -cycloalkyl, -cycloalkenyl, -heterocycl, -aryl, or -heteroaryl. While not wishing to be bound by any theory, it is believed that the phosphorus-containing acid provides an electrophilic hydrogen ion to the amine group to neutralize the amine group in a Lewis acid-base interaction. The transfer of the hydrogen places a positive charge on the nitrogen of the amine group thereby facilitating the formation of an electrostatic interaction with the dissociated phosphorus-containing acid. In neutralizing the basicity of amine functionalities, phosphorus-containing and/or sulfur-containing acid molecules can assist in controlling the pH of the aqueous sizing composition.

In some embodiments, an amine molecule of an acid-amine complex comprises a single amine functionality for association with a phosphorus-containing acid or a sulfur-containing acid. In some embodiments, an amine molecule comprises a plurality of amine functionalities for association with one or a plurality of phosphorus-containing acid and/or sulfur-containing acid molecules.

In some embodiments, for example, molecules of a phosphorous-containing and/or a sulfur-containing acid neutralize substantially all of the amine functionalities of the amine molecules. In other embodiments, molecules of a phosphorus-containing acid and/or a sulfur containing acid neutralize less than substantially all of the amine functionalities of the amine molecules.

In some embodiments, a sizing composition of the present invention comprises stoichiometric ratios of molecules of a phosphorus-containing acid and/or a sulfur-containing acid to amine molecules. In other embodiments, a sizing composition of the present invention comprises non-stoichiometric ratios of molecules of a phosphorus-containing acid and/or a sulfur-containing acid to amine molecules.

In some embodiments, the molar ratio of molecules of phosphorus-containing acid and/or sulfur-containing acid to amine molecules is based on the molar quantity of amine functional groups present in the amine molecules. In such embodiments, for example, the molar ratio of phosphorus-containing acid and/or sulfur containing acid to amine is determined according to Formula (IV):

$$\frac{( moles phosphorus \text{-} containing acid + moles sulfur \text{-} containing acid )}{( moles amine functional groups )} \qquad ( IV )$$

In some embodiments, a molar ratio determined according to Formula (IV) is less than about 1. In some embodiments, a molar ratio determined according to Formula (IV) is less than about 0.75 or less than about 0.5. In some embodiments, a molar ratio determined according to Formula (IV) is less than about 0.25 or less than about 0.10. In other embodiments, a molar ratio determined according to Formula (IV) is greater than about 1. In one embodiment, a molar ratio determined according to Formula (IV) is greater than about 2 or greater than about 3.

In the operation of Formula (IV), for example, an amine molecule can comprise two amine functional groups. Therefore, if one mole of amine molecules are provided in the sizing composition, two moles of amine functional groups are present. Moreover, if one mole of phosphorus-containing acid is added to the sizing composition comprising the amine molecules and no moles of sulfur-containing acid are added, Formula (IV) becomes (1+0)/2, which provides a molar ratio of phosphorus-containing acid to amine of 0.5.

A sizing composition, in some embodiments, comprises an acid-amine component in an amount up to about 100 weight percent on a total solids basis. A sizing composition can comprise an acid-amine component in an amount up to about 80 weight percent, in some embodiments, or up to about 60 weight percent on a total solids basis, in other embodiments. In some embodiments, a sizing composition comprises an acid-amine component in an amount greater than about 1 weight percent on a total solids basis. In some embodiments, a sizing composition comprises an acid-amine component in an amount greater than about 10 weight percent on a total solids basis. In one embodiment, a sizing composition comprises an acid-amine component in an amount greater than 40 weight percent on a total solids basis. In some embodiments, a sizing composition can comprise any amount of acid-amine component effective to impart the desired properties on the sizing composition.

As described herein, acid-amine component of an aqueous sizing composition, in some embodiments, comprises an acid-aminosilane component, the acid-aminosilane component comprising molecules of at least one aminosilane associated with molecules of at least one phosphorus-containing acid and/or sulfur-containing acid. Molecules of at least one aminosilane are associated with molecules of at least one phosphorus-containing acid and/or sulfur containing acid by electrostatic interactions, covalent bonds, dipole-dipole interactions, hydrogen bonds or van der Waals interactions or combinations thereof. In some embodiments, molecules of at least one aminosilane are at least partially neutralized by molecules of at least one phosphorus-containing acid and/or sulfur-containing acid in the formation of the acid-aminosilane component. In one embodiment, for example, a phosphorus-containing acid comprising the acid form of a stabilizer can at least partially neutralize an aminosilane in a manner consistent with that provided in Formula (III) above.

Non-limiting examples of commercially available aminosilanes include A-1100 γ-aminopropyltriethoxysilane, A-1120 N-(β-aminoethyl)-γ-aminopropyltrimethoxysilane, and other aminofunctional silanes in the A-1100 series from OSi Specialties, as well as DYNASYLAN® AMEO 3-aminopropyltriethoxysilane from Degussa AG of Dusseldorf, Germany.

Sizing compositions of the present invention, in some embodiments, can further comprise at least one film former operable to supplement the film forming characteristics of the acid-amine component. In some embodiments, sizing compositions of the present invention can comprise a plurality of film formers. Any film former known to one of skill in the art not inconsistent with the objectives of the present invention can be used. Suitable film formers, in some embodiments, for example, can be compatible with one or more polymeric resins.

The selection of a film former may depend on the polymeric resin to be reinforced to enhance compatibility between the resin and glass fibers coated with a sizing composition comprising the film former. Additionally, selection of a film former may depend on the type of fiber to be sized.

A number of film formers can used in various embodiments of the present invention. Non-limiting examples of film formers for use in various embodiments of the present invention comprise chemically modified polyolefins, polyurethanes, epoxides, or mixtures or aqueous dispersions thereof. In some embodiments, a film former can comprise one or more polyacrylates, polyesters or poly(vinyl acetates).

A film former comprising a chemically modified polyolefin, in some embodiments, is provided as an aqueous emulsion. The term “chemically-modified polyolefin” refers to acid or acid anhydride modified polyolefins either amorphous or crystalline, such as those produced by the method detailed in U.S. Pat. No. 3,416,990, U.S. Pat. No. 3,437,550 and U.S. Pat. No. 3,483,276, each of which is incorporated herein by reference. A discussion of these polyolefins, their modification and emulsification can be found in U.S. Pat. No. 5,130,197, which is incorporated herein by reference.

An example of a crystalline carboxylated polypropylene polymer useful as a film former, in some embodiments of the present invention, is the HERCOPRIME® type resin commercially available from Hercules, Inc. of Bloomington, Del. An example of an amorphous carboxylated polypropylene polymer is EPOLENE® E-43 resin commercially available from the Westlake Chemical Corporation of Longview, Tex. Another suitable film former material is an aqueous emulsion of the EPOLENE E-43 resin, commercially available from Byk-Cera under the trade designation Novacer 1841 emulsion. CHEMCOR 43C30 amorphous carboxylated polypropylene aqueous emulsion commercially available from Chemical Corporation of America is another example of a film former suitable for use in some embodiments. Another commercially available version of an aqueous polyolefin emulsion useful as a film former in some embodiments is the carboxylated amorphous polypropylene from National Starch, Procter Division, sold under the trade designation Protolube RL-5440 polypropylene emulsion. A further suitable film former is an aqueous emulsion of a high molecular weight maleic anhydride grafted polypropylene emulsion commercially available from DSM, B.V. of the Netherlands under the Neoxil 605 trade designation.

Suitable film formers for some embodiments comprise polyurethanes. Polyurethane film forming materials, in some embodiments, are useful for polyamide resin reinforcement applications. In some embodiments, polyurethane film forming compositions are provided as aqueous dispersions such as, for example, the WITCOBOND® series provided by Crompton Corporation-Uniroyal Chemical, including, but not limited to, WITCOBOND® W-290H and WITCOBOND® W-296. Additional examples of commercially available polyurethane aqueous dispersions comprise Aquathane 516 from Reichhold Chemical Company and Hydrosize U2-01 from Hydrosize Technologies, Inc.

In some embodiments, polyurethane film formers comprise aqueous solutions of polyurethane polymers formed by a reaction between an organic isocyanate or polyisocyanate and an organic polyhydroxylated compound or hydroxyl terminated polyether or polyester polymer. The polyurethane dispersion may contain a crosslinking group, in some embodiments.

Another example of a suitable polyurethane is an aqueous emulsion of a polyether-polyurethane NAJ-1037 from Bayer Chemical. Further, the polyurethane may be part of a dispersion comprising a polyurethane and a blocked isocyanate. For example, the following polyurethane/blocked isocyanate emulsions may be suitable for use in the sizing compositions of the present invention: WITCOBOND 60× (Crompton), Baybond 403 (Bayer), Baybond PU-130 (Bayer), Baybond XP-7055 (Bayer), Nopco D641 (Henkel), Neoxil 6158 (DSM), and Vestanat EP-DS-1205 (Degussa).

In some embodiments of the present invention, the at least one film former can comprise epoxide compositions. Epoxide film forming materials, in some embodiments, are useful in polyethylene or polybutylene terephthalate reinforcement applications. Suitable epoxide compositions for use as film formers, according some embodiments, comprise EPON epoxides and EPI-REZ epoxides commercially available Hexion Specialty Chemicals of Columbus, Ohio.

Sizing compositions of the present invention, in some embodiments, comprise at least one film former in an amount up to about 85 weight percent on a total solids basis. In other embodiments, a sizing composition comprises at least one film former in an amount up to about 75 weight percent on a total solids basis. In some embodiments, a sizing composition comprises at least one film former in an amount up to about 60 weight percent on a total solids basis. In some embodiments, a sizing composition comprises at least one film former in an amount up to about 50 weight percent based on a total solids basis. In another embodiment, a sizing composition comprises at least one film former in an amount greater than about 5 weight percent on a total solids basis.

Some embodiments of sizing compositions of the present invention further comprise one or more non-aminofunctional coupling agents in addition to any aminofunctional coupling agents participating in the formation of the acid-amine component. Non-limiting examples of non-aminofunctional coupling agents comprise γ-isocyanatopropyltriethoxysilane, vinyl-trimethoxysilane, vinyl-triethoxysilane, allyl-trimethoxysilane, mercaptopropyltrimethoxysilane, mercaptopropyltriethoxysilane, glycidoxypropyltriethoxysilane, glycidoxypropyltrimethoxysilane, 4,5-epoxycyclohexyl-ethyltrimethoxysilane, chloropropyltriemethoxysilane, and chloropropyltriethoxysilane.

Non-limiting embodiments of sizing compositions of the present invention can also comprise a plurality of coupling agents, some of which may participate in the formation of an acid-amine component and others which do not participate in the formation of an acid-amine component. The use of multiple coupling agents can advantageously result in the sizing composition being compatible with a variety of resins, including thermosetting resins, thermoplastic resins, and other resins. The amount and type of each coupling agent used in a sizing composition of the present invention may be selected based on resin compatibility, effect on fiber glass strand properties (e.g., lower broken filaments, abrasion resistance, strand integrity, and strand friction), and compatibility with other components of the sizing composition.

In some embodiments, a sizing composition of the present invention comprises at least one coupling agent in an amount of up to about 40 weight percent based on a total solids basis. In another embodiment, a sizing composition comprises at least one coupling agent in an amount up to about 25 weight percent or up to about 15 weight percent based on a total solids basis. In some embodiments, a sizing composition comprises at least one coupling agent in an amount up to about 10 weight percent or up to about 5 weight percent on a total solids basis. In other embodiments, a sizing composition comprises at least one coupling agent in an amount up to about 3 weight percent or up to about 1 weight percent on a total solids basis. The foregoing amounts correspond to coupling agent in the sizing composition that does not participate in the formation of the acid-amine component.

In one embodiment, a sizing composition of the present invention does not comprise a coupling agent.

In some embodiments, a sizing composition of the present invention can further comprise at least one lubricant. Any lubricant known to one of skill in the art not inconsistent with the objectives of the present invention can be used. Lubricants can be used, for example, in sizing compositions of the present invention to assist with internal lubrication (e.g., fiber-to-fiber abrasion) and to assist with external lubrication (e.g., glass-to-contact point abrasion). In some embodiments, the at least one lubricant may comprise at least one cationic lubricant. In other embodiments, the at least one lubricant may comprise at least one non-ionic lubricant. In a further embodiment, the at least one lubricant may comprise at least one cationic lubricant and at least one nonionic lubricant.

Cationic lubricants may be used in embodiments of the present invention, for example, to assist with internal lubrication, such as by reducing filament-to-filament or glass-to-glass abrasion. In general, most cationic lubricants known to those of skill in the art can be used in embodiments of the present invention. Non-limiting examples of cationic lubricants suitable for use in sizing compositions of the present invention include lubricants with amine groups, lubricants with ethoxylated amine oxides, and lubricants with ethoxylated fatty amides. A non-limiting example of a lubricant with an amine group is a modified polyethylenimine, e.g. EMERY 6717, which is a partially amidated polyethylenimine commercially available from Pulcra Chemicals of Charlotte, N.C. Another example of a cationic lubricant useful in embodiments of the present invention is ALUBRASPIN 226, which is a partially amidated polyethylenimine commercially available from BASF Corp. of Parsippany, N.J.

In some embodiments, a lubricant comprises one or more partial esters of a branched carboxylic acid copolymer. The partial ester and derivatives thereof are polymers with pendant hydrocarbon and ethoxylated ester chains. A commercially available version of a suitable partial ester of a branched carboxylic acid copolymer is that from Akzo Chemie America of Chicago, Ill. under the trade designation Ketjenlube 522 partial ester. (formerly sold as DAPRAL® GE 202 partial ester.)

Non-ionic lubricants, in some embodiments, comprise at least one wax. Examples of waxes suitable for use in the present invention include polyethylene wax, paraffin wax, polypropylene wax, microcrystalline waxes, and oxidized derivatives of these waxes. One example of a polyethylene wax suitable for use in the present invention is Protolube HD-A, which is a high density polyethylene wax commercially available from Bayer Corporation of Pittsburgh, Pa. An example of a paraffin wax suitable in embodiments of the present invention is Elon PW, which is a paraffin wax emulsion commercially available from Elon Specialties of Concord, N.C.

As provided herein, in some embodiments, lubricants comprising one more amine functionalities can participate in the formation of an acid-amine component.

A sizing composition, in some embodiments, comprises at least one lubricant in an amount up to about 10 weight percent on a total solids basis. In another embodiment a sizing composition comprises at least one lubricant in an amount up to about 5 weight percent on a total solids basis. In a further embodiment, a sizing composition comprises at least one lubricant in an amount less than about 1 weight percent on a total solids basis. In some embodiments, a sizing composition comprises at least one lubricant in an amount of at least 1 weight percent on a total solids basis. The foregoing amounts correspond to lubricant in the sizing composition that does not participate in the formation of the acid-amine component.

In some embodiments, a sizing composition of the present invention can further comprise at least one biocide. A biocide can be added as a precautionary measure to preclude potential problems associated with yeast, mold, aerobic bacteria, and other biological products. Any biocides known to those skilled in the art to control organic growth in sizing compositions for glass fibers can generally be used in embodiments of sizing compositions of the present invention. Non-limiting examples of biocides that can be used in the present invention include organotin biocides, methylene thiocyanate biocides, nitro-biocides, and chlorinated and brominated compounds. A non-limiting example of a commercially available biocide for use in embodiments of sizing compositions of the present invention is Bioban BP-30 from Dow Chemical or Parmetol® DF35 of Schulke and Mayr.

In some embodiments, sizing compositions of the present invention have a pH ranging from about 5.0 to about 10.5. In other embodiments, sizing compositions of the present invention have a pH ranging from about 6.0 to about 8.0. In some embodiments, sizing compositions of the present invention can have a pH less than about 5.0 or greater than about 10.5. The pH of a sizing composition, in some embodiments, is adjusted to fall within any of the foregoing pH ranges.

Some embodiments of the present invention provide a fiber glass strand comprising at least one glass fiber at least partially coated with a sizing composition of the present invention. In some embodiments, a sizing composition of the present invention is a primary sizing composition.

In some embodiments, for example, a sizing composition of the present invention comprising at least one film former and at least one acid-amine component is applied to at least one glass fiber as a primary sizing composition. A sizing composition comprising an acid-amine component without a separate film former is subsequently applied to the at least one glass fiber as a secondary sizing composition. Alternatively, in other embodiments, for example, a sizing composition of the present invention comprising an acid-amine component without a separate film former is applied to at least one glass fiber as a primary sizing, and a sizing composition of the present invention comprising an acid-amine component and at least one film former is subsequently applied to the at least one glass fiber as a secondary sizing.

Fiber glass strands comprising at least one fiber at least partially coated with a sizing composition of the present invention, in some embodiments, can comprise chopped strands. Chopped fiber glass strands, in some embodiments, can have a length ranging from about 3 mm to about 25 mm. In other embodiments, chopped fiber glass strands can have length ranging from about 5 mm to about 25 mm. In another embodiment, chopped fiber glass strands have a length less than about 5 mm or greater than about 25 mm.

Some embodiments of fiber glass strands comprising at least one fiber at least partially coated with a sizing composition of the present invention can comprise continuous strands. Moreover, continuous fiber glass strands, according to some embodiments, can be wound into a single package such as a forming package or a direct draw package. In some embodiments, a plurality of fiber glass strands comprising at least one fiber at least partially coated with a sizing composition of the present invention are assembled into rovings.

Persons of ordinary skill in the art will recognize that the present invention can be implemented in the production, assembly, and application of a number of glass fibers. Any glass fibers known to one of skill in the art not inconsistent with the objectives of the present invention can be used.

Sizing compositions of the present invention can be applied to glass fibers by suitable methods known to one of skill in the art such as, but not limited to, by contacting the glass fibers with a static or dynamic applicator, such as a roller or belt applicator, or by spraying, or by other means. The overall concentration of the non-volatile components in the sizing composition can be adjusted over a wide range according to the means of application to be used, the character of the glass fiber to be sized, and the weight of the dried size coating desired for intended use of the sized glass fibers. In some embodiment, the sizing composition can be applied to glass fibers in the forming operation of the fibers.

The amount of sizing composition on fiber glass may be measured as “loss on ignition” or “LOI”. As used herein, the term “loss on ignition” or “LOI” means the weight percent of dried sizing composition present on the fiber glass as determined by Equation 1:

LOI=100×[(W_{dry}−W_{bare})/W_{dry}] (Eq. 1)

wherein W_{dry} is the weight of the fiber glass plus the weight of the coating after drying in an oven at 220° F. (about 104° C.) for 60 minutes, and W_{bare} is the weight of the bare fiber glass after heating the fiber glass in an oven at 1150° F. (about 621° C.) for 20 minutes and cooling to room temperature in a dessicator.

In some embodiments, a fiber glass strand of the present invention has a LOI ranging from about 0.05 and about 1.5. In another embodiment, a fiber glass strand of the present invention has a LOI ranging from about 0.1 to about 0.5. In a further embodiment, a fiber glass strand of the present invention has a LOI of about 0.4.

Some embodiments of the present invention relate to glass fiber reinforced thermoplastic or thermoset composites. In some embodiments, a glass fiber reinforced thermoplastic or thermoset composite comprises a thermoplastic or thermoset resin and at least one glass fiber at least partially coated with a sizing composition of the present invention. In some embodiments, thermoplastic resins reinforced with glass fibers of the present invention comprise polyolefins such as polyethylene and polypropylene, polyamides, polyphenylene oxide, polystyrenics, or polyesters such as polybutylene terephthalate (PBT) and polyethylene terephthalate (PET). In some embodiments, thermoset resins reinforced with glass fibers of the present invention comprise polyester resins, polyimide resins, phenolic resins, and epoxy resins.

The glass fibers can be used to reinforce polymeric matrices in any of the molding processes known to those skilled in the art utilizing either chopped strand, continuous strand or a mixture thereof. For example, chopped fiber strands can be mixed with a matrix polymer resin melt. The composite melt can be subsequently molded to produce a polymeric part or a shaped device. In some embodiments, glass fibers are mixed with matrix polymer resin in a dry powder mixture.

Glass fibers at least partially coated with a sizing composition of the present invention can have any desired length. In some embodiments, glass fibers at least partially coated with a sizing composition of the present invention have dimensions suitable for use in long fiber reinforcement applications including G-LFT (Granular-Long Fiber Thermoplastic) and D-LFT (Direct-Long Fiber Thermoplastic). In some embodiments of fiber reinforced thermoplastic composites, glass fibers have an aspect ratio ranging from about 5 to 50. As used herein, “aspect ratio” refers to a glass fiber's length divided by the glass fibers diameter. In some embodiments, a glass fiber at least partially coated with a sizing composition of the present invention has an aspect ratio of at least 100 or at least 200.

In some embodiments, a fiber reinforced thermoplastic or thermoset composite comprises any desired amount of glass fibers at least partially coated with a sizing composition of the present invention. In one embodiment, a plurality glass fibers can be present in an amount up to about 90 weight percent of the composite. In another embodiment, the plurality of glass fibers are present in an amount up to about 80 weight percent of the composite. In some embodiments, the plurality of glass fibers are present in an amount up to about 65 weight percent of the composite. In some embodiments, the plurality of glass fibers are present in an amount greater than about 10 weight percent of the composite. In some embodiments, the plurality of glass fibers are present in an amount greater than about 20 weight percent of the composite. In another embodiment, the plurality of glass fibers are present in an amount greater than about 30 weight percent of the composite.

Embodiments of the present invention also provide methods of making a glass fiber reinforced thermoplastic or thermoset composite. In one embodiment, a method of making a glass fiber reinforced thermoplastic or thermoset composite comprises providing a plurality of glass fibers, at least partially coating the plurality of glass fibers with a sizing composition of the present invention, and disposing the plurality of coated glass fibers in a thermoplastic or thermoset resin. As provided herein, glass fibers can be assembled into continuous fiber glass strands. The fiber glass strands can be chopped, remain continuous, or be assembled into rovings for thermoplastic or thermoset reinforcement applications.

Some exemplary embodiments of the present invention will now be illustrated in the following specific, non-limiting examples.

### EXAMPLE 1

A non-limiting embodiment of a sizing composition of the present invention was prepared in accordance with the following formulation:

Sizing Formulation (20 Liters)

| Component | Amount (g) | % of Solids |
|---|---|---|
| Demineralized water | 7000 | 0 |
| Acetic Acid (80%) | 87.2 | 0 |
| Silane¹ | 523.3 | 36.13 |
| Hypophosphorus acid (50%) | 156.0 | 8.67 |
| Film Former² | 1367.5 | 53.20 |
| Lubricant³ | 18.0 | 2.00 |
| Biocide⁴ | 0.018 | 0.0007 |
| Demineralized water to fill | 10847.9 | 0 |

¹DYNASYLAN AMEO 3-aminopropyltriethoxysilane from Degussa AG of Dusseldorf, Germany
²Neoxil 604 from DSM, B.V. of the Netherlands
³DAPRAL GE 202 from Akzo Chemie of Chicago, Illinois
⁴PARMETOL DF35 from Schulke and Mayr

The sizing composition of Example 1 was prepared by providing about 7 liters of demineralized water to a main mixing tank with an agitator. The specified amount of acetic acid was added to the main mixing tank, and the resulting solution was agitated for five minutes. After agitation, the specified amount of aminosilane was added to the main mixing tank, and the resulting solution was agitated for 10 minutes. The specified amount of hypophosphorus acid was subsequently added to the main mixing tank, and the resulting solution was agitated for 10 minutes. The addition of the hypophosphorus acid to the main mixing tank induced the formation of the acid-amine component comprising the aminosilane and the hypophosphorus acid.

The specified amount of film former was subsequently added to the mix tank with agitation. Hot water was added to a premix tank, and the specified amount lubricant was added to the premix tank. The resulting mixture was subsequently agitated for 10 minutes to disperse the lubricant. The lubricant dispersion was then added to the main mix tank. The specified amount of biocide was added to the main mix tank, and demineralized water was added to balance the formulation to 20 liters. The prepared sizing composition had a pH of about 7.5.

### EXAMPLE 2

A non-limiting embodiment of a sizing composition of the present invention was prepared in accordance with the following formulation:

Sizing Formulation (20 Liters)

| Component | Amount (g) | % of Solids |
|---|---|---|
| Demineralized water | 7000 | 0 |
| Acetic Acid (80%) | 87.2 | 0 |
| Silane¹ | 523.3 | 24.07 |
| Hypophosphorus acid (50%) | 156.0 | 5.78 |
| Film Former² | 2655.0 | 68.82 |
| Lubricant³ | 18.0 | 1.33 |
| Biocide⁴ | 0.018 | 0.0005 |
| Demineralized water to fill | 9560.4 | 0 |

¹DYNASYLAN AMEO 3-aminopropyltriethoxysilane from Degussa AG of Dusseldorf, Germany
²Neoxil 604 from DSM, B.V. of the Netherlands
³DAPRAL GE 202 from Akzo Chemie of Chicago, Illinois
⁴PARMETOL DF35 from Sculke and Mayr

The sizing composition of Example 2 was prepared by providing about 7 liters of demineralized water to a main mixing tank with an agitator. The specified amount of acetic acid was added to the main mixing tank, and the resulting solution was agitated for five minutes. After agitation, the specified amount of aminosilane was added to the main mixing tank, and the resulting solution was agitated for 10 minutes. The specified amount of hypophosphorus acid was subsequently added to the main mixing tank, and the resulting solution was agitated for 10 minutes. The addition of the hypophosphorus acid to the main mixing tank induced the formation of the acid-amine component comprising the aminosilane and the hypophosphorus acid, as provided herein.

The specified amount of film former was subsequently added to the mix tank with agitation.

Hot water was added to a premix tank and the specified amount of lubricant was added to the premix tank. The resulting mixture was subsequently agitated for 10 minutes to disperse the lubricant. The lubricant dispersion was then added to the main mix tank. The specified amount of biocide was added to the main mix tank, and demineralized water was added to balance the formulation to 20 liters. The prepared sizing composition had a pH of about 7.5.

### EXAMPLE 3

A non-limiting embodiment of a sizing composition of the present invention was prepared in accordance with the following formulation:

Sizing Formulation (20 Liters)

| Component | Amount (g) | % of Solids |
|---|---|---|
| Demineralized water | 7000 | 0 |
| Acetic Acid (80%) | 139.6 | 0 |
| Silane¹ | 837.4 | 38.25 |
| Hypophosphorus acid (50%) | 156.0 | 5.74 |
| Film Former² | 2124.6 | 54.69 |
| Lubricant³ | 18.0 | 1.32 |
| Biocide⁴ | 0.018 | 0.0005 |
| Demineralized water to fill | 9724.5 | 0 |

¹DYNASYLAN AMEO 3-aminopropyltriethoxysilane from Degussa AG of Dusseldorf, Germany
²Neoxil 604 from DSM, B.V. of the Netherlands
³DAPRAL GE 202 from Akzo Chemie of Chicago, Illinois
⁴PARMETOL DF35 from Sculke and Mayr

The sizing composition of Example 2 was prepared by providing about 7 liters of demineralized water to a main mixing tank with an agitator. The specified amount of acetic acid was added to the main mixing tank, and the resulting solution was agitated for five minutes. After agitation, the specified amount of aminosilane was added to the main mixing tank, and the resulting solution was agitated for 10 minutes. The specified amount of hypophosphorus acid was subsequently added to the main mixing tank, and the resulting solution was agitated for 10 minutes. The addition of the hypophosphorus acid to the main mixing tank induced the formation of the acid-amine component comprising the aminosilane and the hypophosphorus acid as provided herein.

The specified amount of film former was subsequently added to the mix tank with agitation.

Hot water was added to a premix tank and the specified amount of lubricant was added to the premix tank. The resulting mixture was subsequently agitated for 10 minutes to disperse the lubricant. The lubricant dispersion was then added to the main mix tank. The specified amount of biocide was added to the main mix tank, and demineralized water was added to balance the formulation to 20 liters. The prepared sizing composition had a pH of about 7.5.

### EXAMPLE 4

Non-limiting embodiments of sizing compositions of the present invention having the formulations A and B were prepared in accordance with the protocol of Example 1. Each of sizing compositions A and B included a phosphorus-containing acid comprising hypohosphorus acid resulting in the formation of an acid-amine component comprising the aminosilane and the hypophosphorus acid.

Sizing Formulation (20 Liters)

| | | | | | | | mol ratio |
|---|---|---|---|---|---|---|---|
| | Acetic Acid | | Hypo- | | | | of |
| Sizing | (HAc 80%) | Silane¹ | phosphorus | Film Former³ | Lubricant⁴ | | HHP to |
| Composition | (g) | (g) | Acid (HHP) (g)² | (g) | (g) | pH | Silane |
| A | 133.2 | 523.3 | 78.3 | 2600 | 26 | 7.4 | 0.25 |
| B | 88.8 | 523.3 | 156.6 | 2488 | 26 | 7.3 | 0.5 |

¹¹DYNASYLAN AMEO 3-aminopropyltriethoxysilane from Degussa AG of Dusseldorf, Germany (36% solids)
²50% Solution
³Neoxil 605 from DSM, B.V. of the Netherlands (53.2% solids)
⁴DAPRAL GE 202 from Akzo Chemie of Chicago, Illinois (2% solids)

Comparative sizing compositions having the formulations C, D and E were prepared in accordance with the protocol of Example 1. The comparative sizing compositions did not include a phosphorus-containing acid or sulfur-containing acid for formation of an acid-amine component. In sizing compositions D and E, the hypophosphorus acid component was replaced by the acid salt, sodium hypophosphite obtained from Transmare, B.V. of the Netherlands. Sizing composition C did not have an acid content other than the acetic acid.

Sizing Formulation (20 Liters)

| | | | | | | | mol |
|---|---|---|---|---|---|---|---|
| | Acetic | | Sodium | Film | | | ratio of |
| Sizing | Acid (HAc | Silane⁵ | Hypophosphite | Former⁶ | Lubricant⁷ | | SHP to |
| Composition | 80%) (g) | (g) | (SHP) (g) | (g) | (g) | pH | Silane |
| C | 177.6 | 523.3 | 0.0 | 2711 | 26 | 7.0 | 0.0 |
| D | 177.6 | 523.3 | 62.9 | 2562 | 26 | 7.3 | 0.25 |
| E | 177.6 | 523.3 | 125.8 | 2413 | 26 | 7.7 | 0.5 |

⁵¹DYNASYLAN AMEO 3-aminopropyltriethoxysilane from Degussa AG of Dusseldorf, Germany (36% solids)
⁶Neoxil 605 from DSM, B.V. of the Netherlands (53.2% solids)
⁷DAPRAL GE 202 from Akzo Chemie of Chicago, Illinois (2% solids)

Sizing composition A was at least partially applied to continuous fiber glass filaments using a sizing applicator. The fiber glass filaments were gathered into strands and the strands assembled into a continuous roving. The roving was subsequently wound into a forming package on a winder and dried using conventional techniques. The foregoing application, assembly, winding and drying processes were independently repeated for each of sizing compositions B-E to produce independent forming packages of rovings for each sizing composition.

A continuous roving comprising glass fiber filaments at least partially coated with sizing composition A was tested for resistance to broken filaments (fly) according to the following method. The continuous roving was attached to a winding drum and drawn across five tension bars in a fly box for a time period sufficient to draw about 0.5 kg to about 1 kg of continuous roving across the bars. Any broken filaments resulting from drawing of the roving across the tension bars were captured by the fly box. At the completion of the drawing, the broken filaments collected in the fly box were weighed. Moreover, the length of continuous roving drawn over the five tension bars was also weighed. The fly value was subsequently determined according to Formula (V):

[(weight of broken filaments)/(weight of roving drawn)]×1000 mg/kg (V)

The foregoing fly test method was independently repeated for continuous rovings for each forming package corresponding to sizing compositions B-E. The results of the fly testing are provided in Table I.

TABLE I
Fly Testing Results

| Sizing Composition | Mean Fly (mg/kg) |
|---|---|
| A | 5 |
| B | 7 |
| C | 21 |
| D | 19 |
| E | 26 |

As provided in Table I, continuous rovings comprising glass fibers at least partially coated with sizing compositions A or B comprising an acid-amine component demonstrated an order of magnitude less broken filaments in the fly testing in comparison with continuous rovings of sizing compositions C-E. The reduction in the number of broken filaments for sizing compositions A and B provides an indication that the sizing compositions were sufficiently spread over surfaces of the fibers to impart protection of the fibers during the testing process. The desirable spreading properties of sizing compositions A and B comprising an acid-amine component can be attributed to the film forming properties of the acid-amine component. Continuous rovings comprising glass fibers at least partially coated with sizing compositions D or E demonstrated significantly higher numbers of broken filaments. The sizing compositions D and E did not contain an acid-amine component as the salt of a phosphorus-containing acid was used to prepare these sizing compositions.

### EXAMPLE 5

Non-limiting embodiments of sizing compositions of the present invention having the formulations F-K were prepared in accordance with the protocol of Example 1. Each of sizing compositions F-K included a phosphorus-containing acid comprising hypohosphorus acid resulting in the formation of an acid-amine component comprising the aminosilane and the hypophosphorus acid.

Sizing Formulation (20 Liters)

| | Acetic | | | | | | mol |
|---|---|---|---|---|---|---|---|
| | Acid | | Hypo- | Film | | | ratio of |
| Sizing | (HAc | Silane⁸ | phosphorus | Former¹⁰ | Lubricant¹¹ | | HHP to |
| Composition | 80%) (g) | (g) | Acid (g)⁹ | (g) | (g) | pH | Silane |
| F | 130.8 | 523.3 | 78.0 | 2600 | 26 | 7.3 | 0.25 |
| G | 87.2 | 523.3 | 156.0 | 2489 | 26 | 7.4 | 0.50 |
| H | 43.6 | 523.3 | 234.0 | 2377 | 26 | 6.9 | 0.75 |
| I | 209.3 | 837.4 | 78.0 | 2043 | 26 | 8.4 | 0.16 |
| J | 139.6 | 837.4 | 156.0 | 1931 | 26 | 8.9 | 0.31 |
| K | 69.8 | 837.4 | 234.0 | 1820 | 26 | 9.0 | 0.47 |

⁸¹DYNASYLAN AMEO 3-aminopropyltriethoxysilane from Degussa AG of Dusseldorf, Germany (36% solids)
⁹50% Solution
¹⁰Neoxil 605 from DSM, B.V. of the Netherlands (53.2% solids)
¹¹DAPRAL GE 202 from Akzo Chemie of Chicago, Illinois (2% solids)

Comparative sizing compositions having the formulations L-Q were prepared in accordance with the protocols of Example 1. The comparative sizing compositions did not include a phosphorus-containing acid or sulfur-containing acid for formation of an acid-amine component. In sizing compositions L-Q, the hypophosphorus acid component was replaced by the acid salt, sodium hypophosphite obtained from Transmare, B.V. of the Netherlands.

Sizing Formulation (20 Liters)

| | Acetic | | | | | | mol |
|---|---|---|---|---|---|---|---|
| | Acid | | Sodium | Film | | | ratio of |
| Sizing | (HAc | Silane¹² | Hypophosphite | Former¹³ | Lubricant¹⁴ | | SHP to |
| Composition | 80%) (g) | (g) | (SHP) (g) | (g) | (g) | pH | Silane |
| L | 174.4 | 523.3 | 104.1 | 2463 | 26 | 7.7 | 0.41 |
| M | 174.4 | 523.3 | 208.3 | 2217 | 26 | 8.0 | 0.83 |
| N | 174.4 | 523.3 | 312.4 | 1969 | 26 | 8.2 | 1.24 |
| O | 279.1 | 837.4 | 104.1 | 1905 | 26 | 7.9 | 0.26 |
| P | 279.1 | 837.4 | 208.3 | 1660 | 26 | 7.8 | 0.52 |
| Q | 279.1 | 837.4 | 312.4 | 1411 | 26 | 7.7 | 0.78 |

¹²¹DYNASYLAN AMEO 3-aminopropyltriethoxysilane from Degussa AG of Dusseldorf, Germany (36% solids)
¹³Neoxil 605 from DSM, B.V. of the Netherlands (53.2% solids)
¹⁴DAPRAL GE 202 from Akzo Chemie of Chicago, Illinois (2% solids)

Sizing composition F was applied to continuous fiber glass filaments using a sizing applicator, and the continuous fiber glass filaments were gathered into strands. The continuous fiber glass strands were subsequently chopped into 4.5 mm fiber glass strands and dried according to standard techniques. The foregoing application, gathering, chopping and drying processes were repeated for each of sizing compositions G-Q to produce chopped fiber glass strands corresponding to each sizing composition G-Q.

The chopped fiber glass strands of each sizing composition F-Q were subsequently tested for resistance to the formation of fuzz according to the following procedure. 120 g of chopped strands were weighed into a first steel cup having an anti-static cloth placed therein. 120 g of chopped strands were weighed into a second steel cup having a second anti-static cloth placed therein. Both steel cups containing the chopped fiber glass strands were secured into a shaker manufactured by Red Devil Equipment Company of Plymouth, Minn. and subjected to 6 minutes of shaking. Subsequent to shaking, the chopped fiber glass strands of the first steel cup were sieved to separate any fuzz resulting from the shaking treatment. Any fuzz was weighed. The chopped fiber glass strands of the second steel cup were additionally sieved and any resulting fuzz weighed. The % fuzz for chopped fibers glass strands of each steel cup was calculated by dividing the weight of fuzz by 120 g and multiplying the resulting value by 100. The % fuzz for the chopped fiber glass strands of the first and second steel cups was then averaged to produce the final % fuzz value.

The fuzz testing was completed for chopped fiber glass strands of each sizing composition F-Q. The results of the fuzz testing are provided in Table II.

TABLE II
Fuzz Testing Results

| Sizing Composition | % Fuzz |
|---|---|
| F | 0.3 |
| G | 0.5 |
| H | 0.8 |
| I | 1.1 |
| J | 0.8 |
| K | 0.9 |
| L | 3.5 |
| M | 8.3 |
| N | 19.3 |
| O | 10.1 |
| P | 14.9 |
| Q | 16.6 |

As demonstrated in Table II, chopped fiber glass strands comprising glass fibers at least partially coated with sizing compositions of the present invention comprising an acid-amine component (F-K) displayed a % fuzz an order of magnitude and, in some cases, two orders or magnitude less than sizing compositions not containing an acid-amine component.

The reduction in the % fuzz for sizing compositions F-K provides an indication that the sizing compositions were sufficiently spread over surfaces of the fibers to improve the integrity of the chopped fiber glass strands. The desirable spreading properties of sizing compositions F-K comprising an acid-amine component can be attributed to the film forming properties of the acid-amine component. Chopped fiber glass strands comprising glass fibers at least partially coated with one of the sizing compositions L-Q demonstrated a significantly higher % fuzz. The sizing compositions L-Q did not contain an acid-amine component as the salt of a phosphorus-containing acid was used to prepare these sizing compositions.

Desirable characteristics, which can be exhibited by embodiments of the present invention, can include, but are not limited to, providing sizing compositions comprising an acid-amine component having advantageous film forming properties thereby eliminating or reducing the amount of film former in the sizing composition and providing fiber glass strands coated with such sizing compositions which can exhibit reduced numbers of broken filaments and fuzz during processing conditions.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

That which is claimed is:

1. An aqueous sizing composition for glass fibers comprising:
an acid-amine component, the acid-amine component comprising molecules of at least one amine associated with molecules of at least one phosphorus-containing acid or sulfur-containing acid.

2. The aqueous sizing composition of claim 1, wherein the molecules of the at least one amine are associated with the molecules of the at least one phosphorus-containing acid or the at least one sulfur-containing acid by electrostatic interactions, covalent bonds, or combinations thereof.

3. The aqueous sizing composition of claim 1, wherein the molar ratio of the at least one phosphorus-containing acid or sulfur-containing acid to the amine is less than about 1.

4. The aqueous sizing composition of claim 1, wherein the molar ratio of the at least one phosphorus-containing acid or sulfur-containing acid to the amine is less than about 0.75.

5. The aqueous sizing composition of claim 1, wherein the molar ratio of the at least one phosphorus-containing acid or sulfur-containing acid to the amine is less than about 0.5.

6. The aqueous sizing composition of claim 1, wherein the at least one amine comprises an aminosilane, imidazoline, alkylimidazoline, ethoxylate amine oxide, or polyamino fatty acid derivative or a mixture thereof.

7. The aqueous sizing composition of claim 1, wherein the phosphorus-containing acid comprises phosphorus acid, hypophosphorus acid, phosphonic acid, organophosphorus acids, or mixtures thereof.

8. The aqueous sizing composition of claim 1, wherein the phosphorus-containing acid comprises a compound of Formula (I): 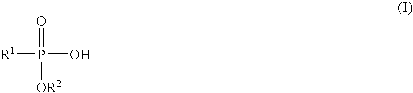
wherein R¹ is -alkyl, -alkenyl, -alkynyl, -cycloalkyl, -cycloalkenyl, -heterocycl, -aryl, or -heteroaryl and R² is -hydrogen, -alkyl, -alkenyl, -alkynyl, -cycloalkyl, -cycloalkenyl, -heterocycl, -aryl, or -heteroaryl.

9. The aqueous sizing composition of claim 1, wherein the phosphorus-containing acid comprises a compound of Formula (II) 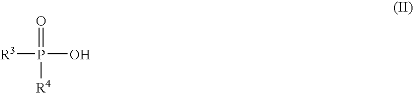
wherein R³ and R⁴ are independently selected from the group consisting of -hydrogen, -alkyl, -alkenyl, -alkynyl, -cycloalkyl, -cycloalkenyl, -heterocycl, -aryl, or -heteroaryl.

10. The aqueous sizing composition of claim 1, wherein the at least one sulfur-containing acid comprises sulfonic acid, organosulfonic acids, hydrogen sulfite or sulfurous acid or mixtures thereof.

11. The aqueous sizing composition of claim 1, wherein the acid-amine component is present in an amount up to about 100 weight percent of the sizing composition on a total solids basis.

12. The aqueous sizing composition of claim 1 further comprising at least one additional acid.

13. The aqueous sizing composition of claim 12, wherein the at least one additional acid comprises a carboxylic acid.

14. The aqueous sizing composition of claim 13, wherein the carboxylic acid comprises formic acid, acetic acid, propionic acid, butyric acid, valeric acid, caproic acid, enanthic acid, caprylic acid, pelargonic acid, capric acid, lauric acid or stearic acid or mixtures thereof.

15. The aqueous sizing composition of claim 1 further comprising at least one film former.

16. The aqueous sizing composition of claim 1 further comprising at least one coupling agent.

17. The aqueous sizing composition of claim 1, wherein the phosphorus atom of the phosphorus-containing acid is not in the oxidation state of 5.

18. The aqueous sizing composition of claim 1, wherein the sulfur atom of the sulfur-containing acid is not in the oxidation state of 6.

19. The aqueous sizing composition of claim 1, wherein the sizing composition is a primary sizing composition.

20. The aqueous sizing composition of claim 1, wherein the sizing composition is a secondary sizing composition.

21. An aqueous sizing composition for glass fibers comprising:
an acid-aminosilane component, the acid-aminosilane component comprising molecules of at least one aminosilane associated with molecules of at least one phosphorus-containing acid or sulfur-containing acid.

22. The aqueous sizing composition of claim 21, wherein the at least one aminosilane comprises γ-aminopropyltrimethoxysilane, γ-aminopropyltriethoxysilane, β-aminoethyltriethoxysilane, N-β-aminoethylamino-propyltrimethoxysilane or 3-aminopropyldimethoxysilane or mixtures thereof.

23. The aqueous sizing composition of claim 21, wherein the molar ratio of the at least one phosphorus-containing acid or sulfur-containing acid to the aminosilane is less than about 1.

24. The aqueous sizing composition of claim 21, wherein the molar ratio of the at least one phosphorus-containing acid or sulfur-containing acid to the aminosilane is less than about 0.75.

25. The aqueous sizing composition of claim 21, wherein the molar ratio of the at least one phosphorus-containing acid or sulfur-containing acid to the aminosilane is less than about 0.5.

26. The aqueous sizing composition of claim 21 further comprising at least one additional acid.

27. The aqueous sizing composition of claim 26, wherein the at least one additional acid comprises a carboxylic acid.

28. A fiber glass strand comprising:
at least one glass fiber at least partially coated with an aqueous sizing composition comprising an acid-amine component, the acid-amine component comprising molecules of at least one amine associated with molecules of at least one phosphorus-containing acid or sulfur-containing acid.

29. A composite material comprising:
a polymeric resin; and
a plurality of glass fibers disposed in the polymeric resin wherein at least one of the plurality of glass fibers is at least partially coated with the aqueous sizing composition of claim 1.

30. The composite material of claim 29, wherein the plurality of glass fibers are present in an amount up to about 90 weight percent of the composite material.

31. The composite material of claim 29, wherein the polymeric resin comprises a thermoplastic.

32. The composite material of claim 31, wherein the thermoplastic comprises a polyolefin, polyamide, polystyrene, polyphenylene oxide, polyester or copolymers or mixtures thereof.

33. The composite material of claim 29, wherein the polymeric resin comprises a thermoset.

34. The composite material of claim 33, wherein the thermoset comprises a polyester, polyurethane, polyimide, polyphenol, expoxy or copolymers or mixtures thereof.

35. A method of making a composite material comprising:
disposing a plurality of coated glass fibers in a polymeric resin, wherein at least one of the plurality of glass fibers is at least partially coated with the aqueous sizing composition of claim 1.
